(19) United States
(12) Patent Application Publication
(10) Pub. No.: US 2021/0236327 A1
(43) Pub. Date: Aug. 5, 2021

(54) MOBILE ITCHING TREATMENT DEVICE WITH INTERFACE

(71) Applicant: DERMAPHARM AG, Grünwald (DE)

(72) Inventor: Daniel BÜNGER VON WURMB, Mönchengladbach (DE)

(73) Assignee: DERMAPHARM AG, Grünwald (DE)

(21) Appl. No.: 15/734,730

(22) PCT Filed: Jun. 6, 2019
(86) PCT No.: PCT/EP2019/064817

§ 371 Date: Dec. 3, 2020

(30) Foreign Application Priority Data
Jun. 7, 2018 (EP) 18176545.4

(51) Int. Cl. A61F 7/00; H04W 4/30; H04W 4/70

(52) U.S. Cl. CPC A61F 7/007; H04W 4/30; H04W 4/70; A61F 2007/0078; A61F 2007/0282; A61F 2007/0086; A61F 2007/0093; A61F 2007/0096; A61F 2007/0052

(57) ABSTRACT

In a first aspect, the invention relates to a device for hyperthermal treatment of itching comprising at least one treatment surface which can be heated by heating at least one heating element to a treatment temperature of 40° C. to 65° C. and can be controlled by a control device, wherein the device exhibits at least one interface and is configured for a power supply of the device by a mobile device and/or for data exchange with a mobile device and/or for control by a mobile device via the interface.

In a further aspect, the invention relates to a system comprising the device and a mobile device, wherein the device is configured for a power supply by the mobile device and/or for data exchange with the mobile device and/or for control by the mobile device via an interface.

In a first aspect, the invention relates to a device for hyperthermal treatment of itching comprising at least one treatment surface which can be heated by heating at least one heating element to a treatment temperature of 40° C. to 65° C. and can be controlled by a control device, wherein the device exhibits at least one interface and is configured for a power supply of the device by a mobile device and/or for data exchange with a mobile device and/or for control by a mobile device via the interface.

In a further aspect, the invention relates to a system comprising the device and a mobile device, wherein the device is configured for a power supply by the mobile device and/or for data exchange with the mobile device and/or for control by the mobile device via an interface.

### BACKGROUND AND STATE OF THE ART

Itching (pruritus) is a subjectively unpleasant sensory perception related to the skin or mucous membrane. It can be localized or affect the whole body. Itching is often accompanied by a burning, stinging or tingling sensation, which person affected often tries to relieve by scratching, rubbing, rubbing, pressing, kneading or rubbing. Therefore, itching is often accompanied by other pathological symptoms of the skin such as scratching, open wounds, crusts and skin infections. Experts assume that itching is mediated by pain receptors in the skin and is transmitted to the brain via the autonomic nervous system. The causes of itching can be very diverse. In addition to dry skin, lack of moisture or allergies, itching can also be caused by external influences and skin irritation, such as bites from mosquitoes or after contact with cnidarians. Itching can be a reaction to chemical, mechanical or thermal stimuli. It may be caused as a result of external irritation, such as the effects of chemical substances, e.g. histamine (mosquito bite), apamine (bee bite), by an allergic immune reaction, by pressure or friction, or by heat or sunlight, wheals, urticaria and other skin reactions associated with itching. From a medical point of view, the causes or underlying diseases leading to itching cover a wide range of dermatological and internal diseases.

For the medicinal treatment of the symptoms of itching a number of drugs or cosmetic products are known. In particular, essential oils such as menthol, thymol or camphor are used extensively to provide short-term cooling. Skin care products such as creams or lotions can also have an analgesic effect by increasing the moisture content of the skin. In addition, antihistamines are helpful therapeutic options, which include the administration of dimetine maleate or mepyramine, for example. Other drugs include topical glucocorticoids, anesthetics, zinc ointments, calcineurin inhibitors or capsaicin. For the treatment of wasps or bee stings, the stinging sites are also treated with ammonia, which, however, only leads to a short-term relief of the itching and only slightly reduces the swelling.

However, it is also known in the state of the art to reduce itching by applying a quantity of heat to the bite of insects. A device for a local, thermal treatment especially of mosquito bites is described in EP 1231875 B1. The device comprises a heating plate with a size of approx. 0.2 cm² which is brought to a temperature between 50° C. and 65° C. while the heating plate contacts the insect bite. This hyperthermic treatment provides lasting relief from itching. On the one hand, the heat application results in a reduction of the thermolabile toxins of the insects, which cause the itching. On the other hand, the heat transfer leads to a masking of the itching by other temperature-dependent skin sensations. Such treatments can thus continue to effectively prevent secondary damage to the skin, for example an inflammation of the insect bite due to scratching. In this way the hyperthermic treatment also effectively reduces the development of wheals accompanying an insect bite.

The possible application hyperthermic treatments also extend to herpes diseases. DE 102005002946 A1 discloses a device for the treatment of herpes diseases. The device comprises a heating plate with a preferred size of 20 mm², which is heated to 49° C.-53° C. for a treatment time of preferably 10-15 sec. During the treatment time, the heating plate contacts the affected skin area of the lips, for example the reddened area or the position where blisters have already formed. On the one hand, the heat application leads to a containment of the multiplication of the causative pathogens by a neutralizing effect on the herpes simplex viruses. On the other hand, the short-term heat treatment leads to a masking of the itching of the herpes disease by stimulating temperature-sensitive nerves. The device is thus characterized by a reduction of the symptoms of the herpes disease such as burning, the appearance of swelling, redness or itching.

From the state of the art US 2007/0049998 A1 also a device for hyperthermic treatment of insect bites is known, which provides a treatment temperature of 50° C. This device has the disadvantage that at this temperature processes that alleviate the itching are not yet or not properly initiated. Processes essential for hyperthermic treatment, which contribute to alleviating the symptoms of insect bites, herpes diseases, jellyfish stings or other diseases associated with itching, are at times only activated in a temperature range between 50° C. and 56° C., especially between 50° C. and 53° C.

The devices for hyperthermic treatment known from the state of the art are characterized by a wide range of possible applications to alleviate the symptoms of insect bites, herpes diseases, jellyfish stings or other diseases associated with itching. However, the devices also have disadvantages.

In particular, no device is known in the state of the art that combines the advantages of modern mobile devices, such as high computing power, networking, but also the ability to serve as an energy source for physical application, with a device for the treatment of pruritus. Mobile devices, especially smartphones, are today not only used for their original purpose, in particular for making phone calls. It is rather common nowadays to combine as many applications as possible in the small portable devices. Today's smartphones are also used as cameras, players, readers, computer games, navigation devices and much more.

In the state of the art, links of mobile control units for the control of treatment devices are known in some cases.

In US 2014/0207219 A1, a stimulator is proposed for the relief of pain in premenstrual syndrome (PMS) using so-called stimulus pods, whereby the stimulus pods can be controlled by remote control in the form of a computer or mobile device.

WO 2015/109397A1 describes a device for the treatment of boils caused by Staphylococcus bacteria with the help of a heat or cooling pad, which can apply ointments or therapeutic solutions. In some cases, wireless software control is to be implemented using mobile devices.

U.S. Pat. No. 9,795,502 B1 discloses a heat mask for the treatment of cold and sinusitis, which can be remotely controlled by a smart phone or tablet.

WO 2018/024753 A1 describes a device for cosmetic treatment of the eye region with alternating temperature application. The control can also be supported by a touch display.

US 2017/0273821 A1 relates to heat pads for cosmetic stimulation of skin or hair, whereby control via a USB interface using a mobile device may also be provided.

For medical applications for the hyperthermic treatment of itching, especially insect bites or herpes, mobile devices have so far played no role. Up to now, there is a lack of devices integrated into or connected to the mobile device, which can cause physical effects.

As a further disadvantage, the known devices may in exceptional cases exceed the desired treatment temperature. In the state of the art it is known to monitor the treatment temperature by means of temperature sensors. However, damage to the device, for example due to the ingress of moisture, can impair the control circuit of the monitoring electronics. This is especially the case if the monitoring of the treatment temperature is integrated into the regular control circuit. In this case, it cannot be excluded that the temperature may rise above the desired treatment temperature. Depending on the contact position of the heating plate or treatment surface, undesirable side effects may occur. Even a short-term temperature increase of more than 65° C. can cause permanent damage to the affected skin areas. This is especially the case for sensitive skin areas, such as lips during a herpes treatment or also thinner skin areas with insect bites.

From US 2007/0049998 A1 a device for hyperthermic treatment of skin complaints is known, which heats a treatment surface via a temperature-controlled heating element to temperatures of 38° C.-67° C. for a duration of at least 5 seconds, typically however for a longer period of time, and uses a fuse to protect against overheating. This type of protection against overheating has the disadvantage that if the fuse is triggered by overheating, it must be replaced. In addition, there is no redundant safety mechanism and if the fuse fails, the treatment surface may overheat for a longer period of time. Furthermore, temperatures above 60° C. or above, especially over a longer period of a few seconds or longer, are perceived as very unpleasant and can lead to skin damage. At the very least, this can jeopardize the success of the treatment, because treatments are stopped early due to an unpleasant skin sensation caused by the high temperatures, thus jeopardizing the success of the therapy. The device is based on the therapeutic idea of destroying germs and killing irritating agents in the skin by applying heat. However, the treatment times and/or temperatures required for this are not suitable for providing lasting relief of itching through targeted stimulation of certain receptors and the modification of the immune system. Temperatures below 42° C. are unsuitable to achieve effects of a therapeutic nature beyond a sensation of heat.

US 2011/0184502 A1 describes a heating pad for various, partly medical applications, which electrically generates temperatures of 38° C.-71° C. for at least several minutes. Non-resetting thermal fuses are proposed in series as a redundant safety feature. Thus, a redundant safety mechanism is available, but it is not reversible and has to be replaced after release. A second disadvantage of using thermal fuses is that they only melt when a temperature above a threshold value is reached. Thus, thermal fuses only react after a certain reaction time when the critical temperature is reached and thus may be too late compared to a fuse. A thermal fuse is triggered by an electric current above a threshold value, which could cause a too high temperature if it flows too long. In addition, both the temperature range and the duration of the heating process are certainly relevant for a large number of applications, but they are unsuitable for sustainably alleviating pruritus by applying heat.

It would therefore be desirable to provide a device that implements the benefits of hyperthermic treatment for the multitude of the above-mentioned diseases and at the same time minimizes safety risks, preferably by using a redundant, but also practical and high standard safety mechanism against overheating.

### Objective of the Invention

An objective of the invention was to eliminate the disadvantages of the prior art. In particular, it was an objective of the invention to combine the advantages of mobile devices offering networking, computing power and the provision of electrical power with the advantages of a device for the treatment of itching.

### SUMMARY OF THE INVENTION

In a first aspect, the invention relates to a device for hyperthermal treatment of itching, comprising at least one treatment surface which can be heated by heating at least one heating element to a treatment temperature of 40° C. to 65° C. and can be controlled by a control device, characterized in that the device exhibits at least one interface and is configured for a power supply to the device by a mobile device and/or for data exchange with a mobile device and/or for control by a mobile device via the interface.

In a further aspect, the invention relates to a system comprising the device and a mobile device, wherein the device is configured for a power supply by the mobile device and/or for data exchange with the mobile device and/or for control by the mobile device via an interface.

### DEFINITIONS AND PREFERRED EMBODIMENTS

In a first aspect, the invention relates to a device for hyperthermal treatment of itching comprising at least one treatment surface which can be heated by heating at least one heating element to a treatment temperature of 40° C. to 65° C. and can be controlled by a control device, characterized in that the device exhibits at least one interface and is configured for a power supply to the device by a mobile device and/or for data exchange with a mobile device and/or for control by a mobile device via the interface.

Tests have shown that the application of heat through such a device can be used surprisingly successfully to treat itching.

To this end the device according to the invention is preferably placed on the affected skin areas. After contacting the skin area with the treatment surface, a control device ensures that the temperature of the treatment surface is regulated in accordance with the invention. The treatment temperature preferably denotes at all times the temperature that is present in the patients skin area. For this purpose, the treatment surface is first heated up to a treatment temperature between 40° C. and 65° C. It is preferred that the heating phase does not require a longer period of time. Preferably, the heating-up phase should not exceed 10 seconds, and particularly preferred not more than 3 seconds. After the heating-up phase, the temperature of the treatment surface is preferably kept at the predetermined treatment temperature. The treatment temperature preferably corresponds to a constant temperature situated in the mentioned range between 40° C. and 65° C.

The treatment temperature is preferably kept constant during the treatment phase. However, it may also be preferred that the treatment temperature is not kept constant. For example, the treatment surface may be guided in a temperature ramp to a maximum temperature in the range of the treatment temperature between 40° C. and 65° C. Subsequently, it may be preferred to lower the temperature below the treatment temperature range for a short time. Afterwards, the temperature may rise again, so that a temporal temperature curve in the form of a ramp is created. The preferred embodiment has surprisingly proven to be more advantageous than maintaining a constant treatment temperature for some skin diseases that cause itching. For example, it can be advantageous to reach the maximum temperature only for a very short time via a rising temperature and allowing for a slight cool down via a ramp.

The treatment phase preferably refers to the period during which the temperature is in the range of the treatment temperature of 40° C. to 65° C. Preferably, the treatment phase lasts from 2 to 20 seconds, particularly preferably the treatment phase lasts between 2 and 12 seconds, particularly preferably between 3 and 6 seconds. It is particularly preferred that the treatment phase designates a continuous period of time. However, it is also possible that the treatment phase is briefly interrupted by temperature control, which exhibits the form of a ramp. In this case, the period of the treatment phase is preferably understood to be the time during which the temperature of the treatment surface is in the range of the treatment temperature of 40° C. to 65° C.

It may be preferable that the treatment phase is not defined in advance, but is individually adapted, for example by the control device, e.g. in conjunction with other externally provided parameters. It may also be preferred that the treatment phase lasts as long as a user presses a control button to maintain treatment. In the following, the treatment phase can be used synonymously with the term treatment duration.

In the sense of the invention, the treatment surface is preferably the area of the device which is heated to the treatment temperature during the treatment and is in direct thermal contact with the skin area. The treatment surface can be a continuous surface. It may also be preferred that the treatment surface consists of several non-contiguous partial surfaces. The size of the treatment surface depends preferably on the disease and the size of the skin areas affected by the symptoms of the itching disease. In the case of insect bites, the size of the treatment surface is between 10 mm² and 100 mm², particularly preferably between 20 mm² and 60 mm². In the treatment of herpes, the treatment surface is preferably between 10 mm² and 80 mm², especially preferably between 20 mm² and 50 mm². Furthermore, it is particularly preferred that the treatment surface for these smaller skin areas is circular. The sizes and geometries of the treatment surface chosen in this way allow for a treatment that is optimally adapted to the cause, which optimizes efficiency and well-being and thus contributing to a more sustainable treatment success.

The size of the treatment surface preferably refers to the total contact surface over which a part of the skin receives a heat impulse. In case of a treatment surface consisting of several partial areas, the size of the treatment surface preferably corresponds to the sum of the individual partial areas. Such a division into partial areas can be advantageous for certain forms of herpes as well as for the treatment of certain parts of the body.

It is preferable that the treatment surface is brought to the desired treatment temperature by means of at least one heating element. In a preferred embodiment, the treatment surface corresponds to the surface of a heating plate, which is heated by means of a heating element, whereby, for example, a semiconductor component can be used. The treatment surface can also refer to a homogeneous material surface, which is tempered by several heating elements. For example, it may be preferable to use two or four heating elements in order to bring the treatment surface particularly homogeneously and quickly to the treatment temperature.

It is preferred that a control device can regulate the heating of the heating element in such a way that the treatment temperature is present at the treatment surface. This ensures optimal control of the treatment temperature and prevents undesired overheating of the treatment surface.

In terms of the invention, the control device is preferably an integrated circuit, processor, processor chip, microprocessor or microcontroller configured to control the temperature of the treatment surface by means of the at least one heating element according to predetermined values for the treatment temperature. Such a control device is characterized by compactness, reliability, cost effectiveness, low power consumption and high control efficiency. The control device can, for example, be included in the device. It may also be preferred that the control device is part of the mobile device, especially if the aim is to have the device controlled by the mobile device via the interface.

The at least one heating element is a component for which different embodiments are sufficiently known from the state of the art. For example, the heating element may include a power resistor, in which a well-defined temperature is generated depending on the current flow. Preferably a field effect transistor (FET) can be used for quantitative control of the current flow through the heating element. However, it may also be preferred to use an FET itself as heating element. In this case the energy dissipation in the transistor is used to generate heat and to bring the treatment surface to the treatment temperature. FETs are particularly preferred as heating elements as their small sizes allow for small dimensioning. Furthermore, FETs are particularly reactive and ensure a very fast response of the heating elements by a very dynamic heat generation and heat release.

The described device preferably comprises a heating element, which may be designed, for example, as an electric heating plate in the form of a round disk with a diameter of about 5 mm. The heating element is preferably fed by a voltage source. A temperature sensor is preferably connected to the heating element. The electrical signal generated by the temperature sensor is advantageously input into the control device, which controls the temperature and the duration of maintaining the maximum temperature of the treatment surface. For example, by pressing a button, the heating-up phase is started by the control device in the heating element. As soon as the electrical signal emitted by the sensor has reached the level corresponding to the specified maximum temperature on the heating element, the treatment phase will preferably begin. For this purpose, a time control is triggered in the control device, which preferably contains a time and temperature control. During the specified duration of the treatment phase, the temperature control primarily ensures that the maximum temperature is maintained and at the same time prevents it from being exceeded. At the end of the treatment phase, the heating process is preferably terminated and the temperature of the heating plate adapts to the ambient temperature. With this device, the treatment surface is preferably heated to a maximum temperature from a range of 40 to 65° C. during the heating phase, whereby a tolerance of ±3° C. is maintained. The maximum temperature in a heating phase can be maintained for a period of preferably 2 to 20 sec, particularly preferably 3 to 20 sec, especially 10 to 15 sec. It may also be preferable to adjust the treatment phase individually.

Preferably, the control device can control which temperature is present at the treatment surface by setting the power supply to the heating element. For example, a calibration can be used to determine the correlation between current flow and/or voltage at the heating element and the temperature at the treatment surface, so that a desired treatment temperature between 40° C. and 65° C. can be set reliably on the basis of the calibration. However, it may also be preferred to regulate the treatment temperature by the control device using a feedback loop. For example, it may be preferable to use a temperature sensor that measures the temperature of the treatment surface, with the control device using the temperature data to regulate the power supply to the heating element. For this purpose, the control device may include a microprocessor that can evaluate measurement data and set current parameters. In this manner the temperature may be controlled very efficiently and reliably.

By regulating the treatment surface at a temperature between 40° C. and 65° C. during a treatment phase, heat is transferred in a well-defined manner. The controlled manner can provide surprisingly effective relief from pain and other unpleasant sensations such as pulling or itching. The effect can also be used in the case of insect bites, where the relief is additionally based on a thermal neutralization of the insects' toxins. On the other hand, the heat causes nerve stimulation, which greatly reduces the subjective perception of pain or itching in the affected areas. The heat transfer leads surprisingly to a masking of the unpleasant sensation by other temperature-dependent skin sensations. Contrary to conventional methods of treating herpes, the method additionally targets a regulation of the pain receptors and activates the free nerve endings of the C-fibers through the preferred heat treatment. The C-fibers refer in particular to the slowly conducting nerve fibers of the somatosensory system and are responsible for the perception of pain. The free ends of the C-fibers, also known as nozic receptors, play a particularly important role in this process. The nerve ends of the fibers are activated by tissue hormones (e.g. histamine, serotonin, substance P). Mast cells near the nerve endings could also be involved in the process by releasing the mediator tryptase. The knowledge about the mechanism of action in herpes is thus exploited to regulate the sensory perception triggered by the fibers in a surprising manner by means of a heat treatment. In addition to the particularly preferred use of the device for the treatment of itching, e.g. after contact with poisonous cnidarians or plants, such as nettles, or in the case of insect bites and stings, the temperature parameters and preferred treatment durations, also allow the treatment of herpes. Furthermore, thermolabile toxins from insect bites can be neutralized.

Furthermore, it was recognized that a particularly strong marking of unpleasant sensations can be achieved if the thermo- and capsaicin receptors TRPV1 and TRPV2 are locally activated simultaneously in the affected skin areas. TRPV1 is involved in acute heat-induced pain in healthy skin and regulates, for example, the sensation of heat at temperatures about 45° to 50° C. In addition, TRPV2 is activated in the case of particularly severe painful heat stimuli that occur at temperatures above 52° C. The activation threshold of TRPV1 is between 40° C. and 45° C., whereas that of TRPV2 is between 50° C. and 53° C. (Yao et al 2011, Somogyi et al 2015, Cohen et al 2014, Mergler et al 2014).

It is preferred that the device exhibits at least one interface and is configured for a power supply of the device by a mobile device and/or for data transmission with a mobile device and/or for control by a mobile device via the interface.

An interface means that the device is configured to interact with and/or be connected to another device. It can be a physical location where the device can be connected to another device. However, it can also be another, optional connection of the device to exchange signals and/or power with another device. This can preferably encompass electrical signals or signals that can be converted into electrical signals by the device. It can also preferably encompass energy, which is exchanged or transmitted. Preferably, it can be electrical energy or energy that can be converted into electrical energy by the device.

The other device is preferably a mobile device. A mobile device can be, for example, a laptop, a cell phone, a smartphone, a tablet computer, a notebook, a Smartwatch and/or a Powerbank.

A connection enabled by such an interface preferably comprises three physical elements: an interface of the mobile device, the interface of the device, and a transmission channel between these two interfaces. The transmission channel can be, for example, a cable-based connection and/or a wireless transmission channel based on the transmission of energy, for example by electromagnetic radiation.

It may also be preferred that the two interfaces are designed in the form of a socket and a matching plug and thus a direct connection can be established, which preferably also enables a mechanically stable connection between device and mobile device. In this case and in case of a cable-based connection possibility the interface would preferably comprise a socket and/or a plug.

Depending on whether the interface is wireless or cable-based or is intended for direct plug and socket connection, the interface comprises differently designed elements.

The interface can be a standardized interface, such as Bluetooth, Lightning, USB, WLAN, etc. However, it can also be an interface developed individually for the device. The interface can be wireless or wired. A cable can be suitable for the transmission of electrical charge carriers, for example a copper cable, but a cable may also be used for the transmission of optical signals, for example a fiber optic cable. A wireless transmission can preferably be based on the transmission of electromagnetic waves in the complete spectral range, for example light signals can be used for wireless transmission, but also short waves, ultrashort waves and/or decimetre waves.

If the interface is e.g. USB, the interface of the device is preferably a USB socket of the device suitable for connection. Furthermore, it is preferably meant that suitable data converters, line drivers, power supplies and/or processors are available on the part of the device in order to use the USB socket as intended, in particular for setting up a USB connection with a mobile device. Furthermore, a connection of electrical components of the device with the interface shall be ensured. This example serves to illustrate an interface of the device and is preferably transferable in principle to other types of interfaces of the device.

An interface can preferably be used for the transmission of data. The device is preferably configured for data transmission with a mobile device via the interface. Data is preferably information that can be output, received and/or processed by an electronic data processing device. A data processing device is preferably a device selected from the group of computers, microcomputers, processors, microprocessors, integrated circuits, smartphones, other mobile devices and/or control devices. Data in digital format, especially in bits, is preferred. However, analog data are also conceivable. Similarly, data can preferably be stored in a memory or on a storage medium.

Data transmission with a mobile device preferably refers to the exchange of data with the mobile device. Data can preferably be received and/or sent by the device. Likewise, data can be stored and/or processed preferentially by the device.

In order to be configured for data transmission via the interface, one data line, preferably several parallel data lines, should be established between the device and the mobile device via the interface. This means that a transmission between mobile device and interface as well as between interface and further electrical components of the device is possible via data lines. The transmission between the mobile device and the interface can preferably take place via an interface of the mobile device, which, depending on the interface, can be wired and/or wireless, as described above. The transmission between the interface of the device and the other components can be realized for example by physical signal lines in the form of cables, which enable the transmission of electrical signals.

It may be preferred that at least one of the elements of the group consisting of mobile device, mobile device interface, device interface and electrical components of the device uses a different data format than at least one of the other elements of this group. It may therefore be preferred that at least one data converter is present at the junctions of elements using different formats, which transmits the data in the direction of transmission into a data format suitable for the following element. If such a data converter is assigned to the interface of the device and/or follows the interface in the direction of the components of the device, the at least one data converter is preferably part of the device.

For sending data, the device and/or its interface preferably includes at least one data sending unit. The data sending unit may transmit data from the device via the interface to the mobile device. The data can preferably be generated by a control device integrated in the device and/or be available on a data memory of the device. Preferably, the data transmission device can also act as a data converter or include such a converter. A person skilled in the art knows suitable devices and/or can set them up routinely.

For receiving data, the device preferably comprises at least one data receiving unit. The data receiving unit can receive data coming from the mobile device via the interface, convert it into a suitable data format if necessary and, for example, forward it in a suitable manner to a control device for further processing and/or a storage medium for storage. An expert knows ways to implement suitable units routinely.

It may also be preferred that the data sending and receiving units are combined in a common unit.

As described above, the device preferably includes a memory that can be used for data transfer. This can be, for example, a memory selected from the group comprising solid state memory, RAM memory, ROM memory, EPROM memory, EEPROM memory, flash memory and/or other memory technologies.

The data can be transmitted in parallel and/or serially. In parallel transmission, several digital information units, so-called bits, can be transmitted simultaneously.

The data may contain preferably parameters related to the treatment, which are transmitted to the control device. These parameters can be treatment parameters such as treatment temperature, treatment duration and/or a time sequence of treatment temperatures to be applied to allow a specific temperature profile over time during treatment. In this regard, metadata may also be transmitted to the control device, for example, from which the control device determines specific treatment parameters. The metadata can encompass, for example, user-related data. For example, gender, age and/or individual treatment preferences of users can be transmitted. The data can also be transferred from the device to the mobile device, for example, the treatment duration and temperature set by the user on the device may be transmitted to the mobile device and linked to user data concerning this user. Further relevant meta data can be generated by a data transfer between the device and a mobile device, for example treatment success, healing process, frequency of application, etc. In addition, the mobile device, which is for example a smartphone, can generate further data, such as GPS position data, time stamps, photos of skin areas affected by itching, etc. These can all be linked and/or correlated with each other, which can generate statistically relevant data, for example concerning successful treatment therapies or a geographically and/or temporally limited occurrence of an insect plague. Also, by linking the mobile device and the device, further interesting applications are conceivable, such as medical treatment and/or consultation by telephone.

The interface can preferably be used for the transmission of electrical energy as well or exclusively. The device is preferably configured for a power supply by means a mobile device via the interface.

To this end it is preferred to enable a power transfer from the mobile device to the device via the interface. The energy transfer relates in particular to the transfer of electrical energy. However, the energy can also be transferred in another form and then converted into electrical energy by the device. The transfer, in particular of electrical energy, can take place via cable. In this case, the interface is formed by providing the possibility of a cable connection between the mobile device or its interface and the device. However, the transmission can also take place in a wireless manner, whereby the device's suitable interface preferably consists of an energy converter, which converts the wirelessly transmitted energy into electrical energy, which can be used to supply the device with power. The transmitted electrical energy received at the interface is preferably transferred to the elements of the device that consume electrical energy, such as the heating element or a possibly integrated control device. The transfer can be realized by suitable connections, e.g. by cables. An energy storage unit, as described below, can be connected in between and/or downstream. The use of suitable voltage regulators and/or charge regulators to supply and/or charge the energy storage unit and the electrical connection of individual elements of the device to the interface is also preferred and can be routinely carried out by a specialist.

Preferably the transfer of energy and data, can take place in parallel. It may also be preferable to use interface only for one kind of transfer, for example to transmit electrical energy. Some interfaces, such as USB, provide for parallel transmission of power and data. Even with a wireless interface, electrical energy and/or data can be transmitted, preferably in parallel, for example by induction coils or by electromagnetic radiation, which is converted into electrical voltage by solar cells and/or photodiodes.

The device is preferably configured for control by a mobile device via the interface.

Control is preferably understood to mean controlling the heating of the heating element while monitoring the treatment parameters, such as treatment temperature and treatment duration. To this end measurement data of the temperature sensor and/or time information should be used. It may be preferred that the device itself does not comprise a control device and the task of the control device is taken over by a suitable hardware and/or software of the mobile device. In this case it is preferred that the mobile device includes a control device. It may be preferred, for example, that the control signals, e.g. for heating the heating element, are transmitted directly from the mobile device via the interface to the mobile device. The control can for example be accomplished by controlling the transmitted current and/or by a pulse width modulated signal from the mobile device. In the same manner, the measurement data of the temperature sensor can preferably be transmitted directly to the mobile device via the interface and read out by the mobile device directly or after a suitable signal conversion and used for control.

It may also be preferred that the device includes a rudimentary processor, such as a microprocessor and/or a data converter, which converts the control commands of the mobile device into suitable signals to control the heating element. Similarly, signals from the temperature sensor can be converted into signals suitable for the mobile device and transmitted to the mobile device via the interface.

For the transmission of the control data via the interface, the data transmission described above may be preferably used.

For control by a mobile device via the interface, the device preferably exhibits at least one electrical connection between interface and processor and/or heating element and/or temperature sensor. The electrical connection can be in the form of an electrical cable. Depending on the implementation, data converters are provided between the interface and the connected elements mentioned.

In this manner the device may be kept particularly simple, inexpensive and compact. Existing control resources of the mobile device, for example of a smartphone, can be used. It can also be advantageous that the device includes a control device and only treatment parameters are transferred from the mobile device to the control device. Such a transfer can optionally be made before each treatment, but it may also be preferable that a transfer only takes place, when new treatment parameters are to be used.

It is preferable that the device includes the control device. The control device may have the above-mentioned characteristics and can act in the described manner to control the heating element.

In a preferred embodiment, the device is configured for parameterization of the control device by the mobile device. In this configuration, user parameters are transferred from the mobile device via the interface to the control device of the device. These parameters may include in particular a treatment temperature and treatment duration. Thus, parameters suitable for the respective user and his or her condition can be transmitted individually before each treatment. A particularly individual treatment can be carried out and the success of the treatment can be increased. Preferred parameters of individual users can also be exchanged via the mobile device. If, for example, a user has had particularly good experience with a certain treatment temperature and treatment duration in the case of mosquito bites, he can make the relevant parameters available to other users. These can be exchanged via networked mobile devices. Common applications such as e-mail, WhatsApp or Facebook can be used to this end. For parameterization, data transfer between mobile device and the device via the interface is preferred.

In a preferred embodiment, the device is configured for a transmission of a control program of the control device by the mobile device. The control program preferably includes an algorithm that determines how the treatment temperature and/or treatment duration of the device is controlled. The control program may, for example, include certain control algorithms. In addition to control devices whose control algorithm cannot be changed after a single setting, as is the case with some microprocessors, control devices can be used which can be programmed several times. The program can be software-based and/or hardware-based. For example, a Field Programmable Gate Array (FPGA) can be used as a control device, which can be completely rewired by a transferred control program. Its logical circuits, which together form the control program, can be reprogrammed. This reprogramming can be done when the device is connected to a mobile device via the interface by transferring a control program through the mobile device and loading it onto the FPGA module. In this way a control program can be updated and improved regularly without having to replace the entire device. For the transfer of a control program, a data transfer between mobile device and device via the interface is preferred.

It may be preferred that the mobile device includes the control device. The operation, implementation and advantages of this embodiment have already been described above.

In all the above-mentioned embodiments, the device can be kept compact and, for example, can be directly coupled to the mobile device, e.g. a smartphone, via an interface. In this case, a user can use the device through his smartphone, for example by plugging the device onto the smartphone, whereby a mechanical connection is preferably effected by the coupling of the interfaces themselves, and the smartphone can be used to control and/or as a handle of the device. In the case of wireless interfaces, the device itself can be shaped in such a way that it can be mechanically connected to the mobile device, for example by plugging it on, and therefore a connection is also possible without a wired interface. Such a device is very practical and can be kept extremely small. In this regard it may be seen as an advantage that a user usually carries his mobile device, especially his smartphone, with him permanently and can use the device in connection with the mobile device.

However, it is also possible that the device itself is used autonomously, for example if a separate control device is available. If a compact energy storage unit is also available, the device can function autonomously, but at the same time be kept very compact.

In a preferred embodiment of the device, the power supply of the device by the mobile device and/or the data exchange with the mobile device and/or the control by the mobile device via the interface is wireless.

For a wireless control as well as for wireless data transmission, a wireless transmission of signals via the interface is preferred. The wireless transmission can take place, for example, in the form of electromagnetic waves, in particular by transmission of light signals. To this end the interface preferably comprises at least one receiving unit and/or one sending unit. A receiving unit may for example include a suitable photodetector such as a photodiode. In addition, other optical elements such as lenses could be included, which a person skilled in the art can provide for a free beam optical transmission. For example, a suitable light source such as a laser and/or an (organic) LED could be used as a transmitter. Furthermore, a person skilled in the art may use suitable optical elements, e.g. for collimation of the transmitted light beam. Likewise, suitable converters and/or drivers can preferably be used for conversion between optical signals and suitable internal electrical signals of the device.

Furthermore, an electromagnetic signal can preferably be based on electromagnetic waves in the non-visible spectral range, for example (near) infrared signals, short waves, ultra-short waves and/or decimeter waves can be used. Also in this regard a person skilled in the art would know how to design an interface with suitable transmitters and/or receiving antennas, drivers and data converters as well as interconnections between the interface and other electrical elements of the device.

For a power supply of the device by a mobile device via the interface, energy is preferably transmitted, for example in the form of electromagnetic waves, which is then converted into usable electrical energy in the device and is preferably transferred to the electrical elements of the device and/or an energy storage unit. A wireless power supply is described in detail below.

In a preferred embodiment of the device, the treatment temperature is 40° C. to 60° C. and the size of the treatment surface is between 1 cm² and 18 cm². The embodiment allows for a particularly reliable treatment of large pruritus areas. In the past, experts were of the opinion that the treatment of large-area skin conditions could not be treated by heat input, since otherwise the total heat transferred to the skin would be too great. However, it was recognized by the inventors that with the disclosed treatment surfaces and treatment temperatures an effective treatment of pruritus over a large area is possible. It was also surprising that a device that has a relatively large treatment surface can be connected to a mobile device in the above-mentioned manner. An increased energy demand can be surprisingly compensated by a power supply from a mobile device.

In another preferred embodiment of the device, the treatment temperature is 40° C. to 60° C. and the size of the treatment surface is less than 1 cm². Such a device is particularly suitable for the treatment of herpes, especially on the lips, and mosquito bites. At the same time such a device can be kept very compact. The data obtained from the treatment of mosquito bites from a large number of users and preferably from a large number of devices can be used advantageously for statistical analysis. For example, mosquito plagues can be detected and predicted.

In a preferred embodiment, the mobile device is selected from the group comprising PC, laptop, desktop PC, cell phone, smartphone, tablet computer, personal digital assistant (PDA), notebook, subnotebook, smartphone, walkman, discman, MP3 player, pocket TV (portable TV), e-book reader, portable electronic media output device, GPS device, portable satellite communication interface device, handheld, pocket computer (‘pocket computer’), mobile computer, camera, video camera, wristwatch, calculator, television, MacBook, iPhone, iPad, iPod, iMac, Mac mini, Mac Pro, Smartwatch and/or Powerbank.

These mobile devices have proven to be particular practical for use with the device. Especially mobile devices, which have an inherent high computing power and a high flexibility regarding their application, can form a particularly powerful system in combination with the device. Such devices are represented for example by smartphones. With smartphones, surprising advantages can be achieved by application programs (so-called apps) individually tailored to the device. For example, networked user data can be used to make statistically relevant statements on promising treatments, which, if a sufficiently large number of data is evaluated, can go far beyond the significance of a particular medical study. In this way, the success of the treatment can be increased in a surprisingly strong manner. Similarly, synergistic effects can be achieved by predicting insect plagues based on user data obtained from past uses of the device. Since the device can now not only treat insect bites but also prevent them synergistic effects are achieved. Furthermore, an improved medical treatment of itching due to an analysis of individual user data at a medical specialist is possible.

In a preferred embodiment, the device includes at least one energy storage unit. Energy storage can be realized for example by at least one accumulator, a battery and/or a capacitor. Preferably an electrical energy storage unit is meant. A battery is an energy storage unit, preferably an electrochemical energy storage unit, whose stored energy can preferably not be recharged after consumption. Examples include zinc-manganese batteries, alkaline manganese batteries, zinc chloride batteries, zinc-carbon batteries, zinc-air batteries, mercury oxide-zinc batteries, silver oxide-zinc batteries, nickel oxyhydroxide batteries, lithium batteries; lithium iron sulphide batteries, aluminium-air batteries, bio-batteries, e.g. based on magnesium/NaCl/iron+molybdenum+tungsten and/or Edison-Lalande elements. Batteries may also include button cell batteries.

A capacitor is preferably an electrical component that stores electrical charge in an electric field.

In particular, the energy storage unit may be at least one accumulator.

In contrast to batteries, accumulators or rechargeable batteries are preferably rechargeable. Accumulators may include for example lithium ion batteries, lithium cobalt dioxide batteries, lithium polymer batteries, lithium manganese batteries, lithium iron phosphate batteries, lithium iron yttrium phosphate batteries, lithium titanate batteries, lithium metal polymer batteries, lithium air batteries, lithium sulfur batteries, sodium nickel chloride high temperature batteries, Sodium-sulfur batteries, sodium-ion batteries, nickel-cadmium batteries, nickel-iron batteries, nickel-hydrogen batteries, nickel-metal hydride batteries, nickel-zinc batteries, lead batteries, PTMA batteries, rechargeable alkaline manganese batteries, tin-sulfur-lithium batteries, silver-zinc batteries, vanadium redox batteries and/or zinc-bromine batteries, silicon air batteries. Lithium-polymer and/or metal-hydride batteries can also be used, which are particularly flexible and adapted to the applications and particularly powerful.

The aforementioned embodiment of the device has the advantage that the device is supplied with electrical energy both when the interface is connected to a mobile device and also independently of such connection. In particular the embodiment comprising at least one accumulator (or possibly a capacitor) may be supplied with electrical energy and charged when the interface is connected a mobile device. The accumulator may be dimensioned in such a manner that it allows for at least one application without a connected interface with the mobile device. Thereby the device does not depend on a connection to the mobile device for its use and may be used more flexible.

In a preferred embodiment, the accumulator includes a solid state accumulator, preferably a lithium ceramic accumulator. The embodiment is characterized by a particularly high level of safety and is preferably usable even after partial destruction.

In a preferred embodiment, the device is configured for power supply and/or charging of the energy storage unit by wireless energy transmission, preferably via the interface. In wireless energy transfer, the electrical energy is preferably transferred without a connection between the device to be charged and the mobile device via an electrical cable. The electrical energy can be transmitted by electromagnetic fields, for example. Inductive coupling through at least one coil each in the mobile device and in the device can be used to this end. It can also be advantageous to capacitively couple the device and the mobile device through at least one capacitor plate each. Even over long distances, so-called far-field transmission can be used to transmit preferentially electrical energy contained in electromagnetic waves between a transmitter and receiver. Likewise, light emitted by the mobile device can also transmit energy, which is converted into electrical voltage in at least one solar cell of the device. It may also be preferable to transmit electrical energy directly between conductive surfaces of the mobile device and the device via a contact between these surfaces. The transferred energy can be used directly to heat the heating element or preferably to charge an accumulator integrated in the device.

A person skilled in the art knows how to equip the interface or device with suitable elements to this end, such as at least one induction coil, a capacitor plate, an antenna, a solar cell, a photodiode, a charge controller and/or a voltage regulator, and how to interconnect the suitable elements to realize the wireless energy transmission.

A wireless interface allows for an energy transmission supply without the need for cumbersome cables between the device and the mobile device. Since a wireless interface can also be used for data transmission, parameterization, transmission of a control program and/or for control, the use of cables can be completely avoided. A mobile device can be used preferably as a sender for wireless power transmission, which serves only this purpose and is plugged directly into a socket.

In a preferred embodiment, the device is configured for power supply and/or charging of the energy storage unit by a cable, especially via the interface. In this configuration, the device, in particular the interface, comprises a cable or a plug and/or a socket for connection to a cable. Furthermore, it is preferred that the electrical energy within the device is transferred to suitable elements, in particular to the energy storage unit by means of connections. A voltage regulator and/or charge regulator is advantageously connected in between. A person skilled in the art knows suitable elements and their interconnections. A transmission of electrical energy in this way is particularly robust, energy efficient and cost-effective.

In a preferred embodiment an interface is selected from the group consisting of Bluetooth, Lightning, Jack plug, Coaxial plug, Apple 30-pin dock connector, ASUS Media Bus proprietary, CAMAC, EISA, ISA, LPC, MBus, MCA, Multibus for industrial systems, NuBus or IEEE 1196, OPTi local bus, PCI, ATA, PATA, IDE, EIDE, ATAPI, S-100 bus or IEEE 696, SBus or IEEE 1496, SS-50 bus, Runway bus, GSC/HSC, Precision Bus, STEbus, STD Bus, Unibus, Q-Bus, VLB or VL-bus, VMEbus, PC/104, PC/104-Plus, PCI-104, PCl/104-Express, PCl/104, Zorro II and Zorro III, 1-Wire, HyperTransport, I²C, PCIe, SATA, SPI bus, UNI/O, SMBus, IrDA, WLAN, ZigBee, NFC, Wibree, WiMAX, IrDA, optical radio relay, eBus, USB, Micro USB, Type C and/or FireWire. By providing an interface selected from this group, a high flexibility regarding the mobile device used and the available interface is provided. Furthermore, these interfaces have proven suitable for a multitude of different applications. A person skilled in the art knows how to design such an interface. For example, a person skilled in the art would know that he would have to install a suitable socket and/or plug for a Lightning interface, and he would also know that he would have to use suitable processors, e.g. host controllers. The person skilled in the art would also know how to connect the electrical elements of the device to the interface.

For example, with a WLAN interface the person skilled in the art would also know that he would have to install at least one suitable antenna in the device to send and/or receive signals. The selection of suitable control processors, data converters and/or controllers as well as an interconnection of the interface within the device can also be routinely carried out by the person skilled in the art.

It may be preferable to use the interface to connect the device directly to an electrical outlet for power and/or charging. In a preferred embodiment, it is possible, for example, to draw electrical energy directly from the socket using a standard USB charger.

In a preferred embodiment, the device comprises at least one first temperature sensor for measuring the temperature of the treatment surface and the regulating device and/or control device adjusts the temperature of the at least one heating element based on the measurement data of the temperature sensor.

In the sense of the invention, a temperature sensor is preferably an electrical or electronic component which generates an electrical signal depending on the temperature at the sensor. A large number of temperature sensors are known in the state of the art, such as semiconductor temperature sensors, resistance temperature sensors, pyroelectric materials, thermocouples or oscillating crystals. The control device is preferably configured in such a manner that it can record and evaluate the measured values of the temperature sensors in order to regulate the heating plates. The regulation of the heating plates can preferably be accomplished by applying an electric current or voltage. It is particularly preferred that the temperature sensor measures the temperature of the treatment surface directly, i.e. that the temperature sensor is in contact with the treatment surface, wherein the temperature sensor may be present on the internal side of the treatment surface as well as on the external side of the treatment surface or be implemented in the treatment surface. However, it may also be preferred that the temperature sensor does not contact and monitor the treatment surface directly, but instead the heating elements or a material point between the heating elements and the treatment surface. In case of several heating elements heating the treatment surface, it may be preferred to place the temperature sensor between the heating elements. Likewise, a conclusion may be drawn regarding the temperature of the treatment surface from the measurement data for the temperature across the heating elements or a measuring point at a certain distance from the treatment surface. In the sense of the invention, it is preferred that the temperature of the treatment surface relates to the average temperature of the treatment surface.

Evaluation of the temperature of the treatment surface allows a particularly precise regulation of at least one heating element to ensure an optimal temperature distribution across the treatment surface and thus a heat transfer to the skin areas to be treated. Especially with regard to the manifold application possibilities of the device for the treatment of different diseases, which can be accompanied by itching, a temperature-based feedback regulation with the aid of the control device is suitable for performing a reliable hyperthermic treatment with optimal temperature values. Such a device for controlling the treatment temperature is particularly simple, robust and cost-effective.

In a preferred embodiment, the heating element heats the treatment surface to a treatment temperature of 40° C.-65° C. The provision of a treatment temperature of 40° C.-65° C. represents a surprisingly improved treatment of itching.

In a preferred embodiment, the heating element heats the treatment surface to a treatment temperature of 50 to 65° C., preferably 55 to 60° C. There were misconceptions of the experts regarding the treatment temperatures of itching, which could be overcome with the provided treatment temperature. It was also surprising that through an interface with a mobile device sufficient energy could be provided for said preferred treatment temperature.

In a preferred embodiment, the heating element heats the treatment surface to a treatment temperature of 42° C.-56° C. The performance regarding the treatment of itching was surprisingly increased by the temperature range provided in this embodiment.

In a preferred embodiment, the heating element heats the treatment surface to a treatment temperature of 42° C.-53° C. Thus, additional means for the treatment of itching can be provided.

In a preferred embodiment, the heating element heats the treatment surface to a treatment temperature of 43° C.-47° C. The effectiveness of these treatment temperatures in treating itching is surprisingly high. At the same time, the electrical effectiveness in terms of energy consumption is also greater than in other temperature ranges. This allows the overall effectiveness to be increased synergistically.

In a preferred embodiment, the heating element heats the treatment surface to a treatment temperature of 50° C.-53° C. The temperature range has proven to be particularly effective and popular with test persons, so that a great economic success is foreseeable.

In a preferred embodiment, the heating element heats the treatment surface to a treatment temperature of 50° C.-55° C. In this temperature range the yield of heat generated per consumed energy is particularly high.

In a preferred embodiment, the heating element heats the treatment surface to a treatment temperature of 55° C.-60° C. The treatment temperature in this range is surprisingly effective. This proved to be lucky choice, because the area that proved to be particularly effective against itching was selected from a wide range of parameters.

In a preferred embodiment, the heating element heats the treatment surface to a treatment temperature of 60° C.-65° C. This temperature range was chosen contrary to the trend in scientific technology.

In a preferred embodiment, the treatment temperature is maintained for a treatment period of 1-60 sec. In this manner, the technical possibilities for the treatment of itching with heat can be increased.

In a preferred embodiment, the treatment temperature is maintained for a treatment period of 1-10 sec. Such a short treatment duration yields a saving of time in the treatment of itching.

In a preferred embodiment, the treatment temperature is maintained for a treatment period of 10-20 sec. This treatment duration has proven to be particularly reliable in the treatment of itching.

In a preferred embodiment, the treatment temperature is maintained for a treatment period of 20-30 sec. Surprisingly, this can provide a second way of treating itching with heat.

In a preferred embodiment, the treatment temperature is maintained for a treatment period of 30-40 sec. It has been shown that with such a treatment duration the yield of the released heat quantity is particularly high.

In a preferred embodiment, the treatment temperature is maintained for a treatment period of 40-50 sec. There have been some misconceptions amongst experts of the field about the appropriate treatment duration for hyperthermic treatment of itching, which have been overcome by this treatment duration.

In a preferred embodiment, the treatment temperature is maintained for a treatment period of 50-60 sec. Errors in the treatment of itching can thus be avoided.

In a preferred embodiment, the treatment temperature is maintained for a treatment period of 2-20 sec. The treatment duration represents a decisive improvement over known methods for the treatment of itching.

In a preferred embodiment, the treatment temperature is maintained for a treatment period of 2-12 sec. From a variety of imaginable or known treatment durations, the treatment duration is a lucky choice, which is was not foreseeable in terms of effectiveness in the treatment of itching.

In a preferred embodiment, the treatment temperature is maintained for a treatment period of 3 to 6 seconds. The efficiency of the device can be increased by this short and at the same time particularly effective treatment duration.

In a preferred embodiment, the treatment temperature is maintained for a treatment period of 4-6 sec. This treatment time represents a decisive departure from the usual technical standards.

In a preferred embodiment, the heating element heats the treatment surface to a treatment temperature of 50 to 65° C., preferably 55 to 60° C., and the treatment temperature is maintained for a treatment period of 2 to 12 sec, preferably 3 to 6 sec. Tests have shown that if the treatment is applied immediately after the bite, mosquito bites can be successfully inactivated at a maximum temperature of 55° C. The exposure time is not critical at a temperature of 55° C., so that the treatment can be repeated several times at this temperature if necessary. Furthermore, due to the anesthetic effect of insect venom, heat treatment of the skin at the bite site is perceived as significantly less unpleasant than in other areas.

In a preferred embodiment, the heating element heats the treatment surface to a treatment temperature between 42° C. and 56° C. and the treatment temperature is maintained for a treatment period of 2 sec to 20 sec.

The embodiment can provide surprisingly effective relief from pain and other unpleasant sensations such as pulling or itching. The heat impulse causes nerve stimulation, which greatly reduces the subjective perception of pain or itching in the affected areas. The heat transfer leads to a surprising masking of the unpleasant sensation by other temperature-dependent skin sensations. Contrary to conventional methods of treating herpes, the method additionally targets a regulation of the pain receptors and activates the free nerve endings of the C-fibers through the preferred heat treatment. The knowledge about the mechanism of action in herpes is thus exploited to regulate the sensory perception triggered by the fibers in a surprising manner through a heat treatment. In addition, the mentioned treatment durations and temperature parameters allow for a use of the device for herpes treatment. Likewise, itching disorders, e.g. after contact with poisonous cnidarians or plants, such as nettles or with insect bites and stings, can be treated. Even thermolabile toxins from insect bites can be neutralized.

Furthermore, it was recognized that the combinations can achieve a particularly strong masking of unpleasant sensations, since the thermo- and capsaicin receptors TRPV1 and TRPV2 are locally activated simultaneously in the affected skin areas. A person skilled in the art, even with knowledge of the literature, would not assume that it is precisely the activation of these receptors that enables a particularly effective masking of the unpleasant sensations, which also works in the treatment of herpes. This is a surprising finding.

This combination of treatment temperature and treatment duration can also increase the technical possibilities for treating itching.

In a preferred embodiment, the heating element heats the treatment surface to a treatment temperature of 49° C. to 53° C. and the treatment temperature is maintained for a treatment period of 3 to 20 sec. The preferred treatment temperatures and durations are surprisingly suitable for treating virus-induced herpes skin diseases in the form of a rash accompanied by blistering, adjacent skin areas or diseased skin areas recognizable by the first signs of herpes, without causing painful sensations or even burns at the same time. Avoidance of painful sensations and burns are particularly important in the sensitive area of the mouth, especially the lips. Even in the treatment of itching, the combination mentioned above can achieve a surprising increase in the effectiveness of the treatment.

In a preferred embodiment, the heating element heats the treatment surface to a treatment temperature between 40° C. and 65° C. and the treatment temperature is maintained for a treatment period of 4 sec to 6 sec.

By regulating the treatment surface at a temperature between 40° C. and 65° C. for a treatment phase between 2 s and 12 s, preferably between 4 s and 12 s, particularly preferably between 4 s-6 s, a heat impulse is generated which allows a well-defined amount of heat to be applied to the skin area in a controlled manner. In this way an effective treatment can be provided while saving time.

In a preferred embodiment, the heating element heats the treatment surface to a treatment temperature between 42° C. and 53° C., particularly preferably between 50° C. and 53° C., and the treatment temperature is maintained for a treatment duration of 4 sec to 6 sec. It has been shown quite surprisingly that the above-mentioned parameters can reduce itching especially on large areas of skin. A combination of a treatment temperature of 42° C. and 53° C. and especially with the preferred treatment temperature between 50° C. and 53° C. allows for an effect on the skin areas that quickly and effectively relieves itching. It has been recognized that a particularly strong masking of an itching sensation can be achieved, since the thermo- and capsaicin receptors TRPV1 and TRPV2 are locally activated simultaneously in the affected skin areas.

The preferred embodiment, which provides for temperature control of the treatment surface within a narrow range between 50° C. and 53° C., allows simultaneous activation of the receptors in a surprisingly effective manner without causing unpleasant strong pain sensations in the persons to be treated. Experimental tests have shown that the activation threshold range of TRPV2 represents an optimized range. It is expected that this leads to a feedback mechanism between the receptors, which masks the itching particularly effectively without causing side effects. A person skilled in the art could not expect the effect. Rather, a person skilled in the art would have assumed that at a treatment temperature of 50° C.-53° C. over a period of 4 s to 6 s, severe skin irritation or pains up to slight burns may occur. Instead, the preferred treatment of the skin areas leads to a reduction of the itching sensation, which continues for hours after the treatment. The long-lasting effect of the preferred embodiment of treatment may be attributed at least in part to an immunoregulation through the heat transfer. Thus, not only the sensation of pain is masked, but the local irritation of the skin is actively suppressed by a regulation of the immune system. Advantageously, a single treatment can therefore lead to a lasting elevation of the itching sensation. However, it may also be preferable to carry out a treatment several times in chronological order. The interval-like transfer of heat with a treatment phase of 4 s to 6 s achieves an optimal effect on the signal pathways of the pruritus without triggering undesirable side effects. Surprisingly, especially for the preferred treatment duration of 4 s-6 s and the intended treatment temperature, especially for the particularly preferred treatment temperature of 50° C.-53° C., it has been found that an exceptionally effective activation of TRPV1 and especially TRPV2 receptors takes place. It was quite surprising that the specific selection of treatment temperatures and durations was particularly effective in reducing itching. The C-fibers, especially their free ends, are moreover addressed by the preferred selection.

In a preferred embodiment, the size of the treatment surface is between 0.1 cm² and 18 cm². Thus, the technical possibilities for the treatment of itching can be increased.

In a preferred embodiment, the size of the treatment surface is between 1 cm² and 18 cm². With a treatment surface of this size, a treatment success can be achieved in a short time in the treatment of itching.

In a preferred embodiment, the size of the treatment surface is at least 6 cm², preferably at least 7 cm².

External stimuli of chemical, mechanical or physical nature, which can trigger itching, are perceived by three different receptor cells (sensory cells). These sensory cells involve what are called open nerve endings, whose stimulus-receiving structures are located in the epidermis and the underlying dermis, and whose axons transmit the signals via perceived stimuli to the spinal cord. The non-myelinated C-fibers are of particular importance for these sensory cells. Their receptive structures are partially located up to 0.1 mm below the skin surface. C-fibers are divided into polymodal mechanically and heat-sensitive fibers and mechanically insensitive C-fibers, which can also be stimulated by heat. C-fibers not only perceive pruritogenic stimuli, but also serve as nociceptors (pain receptors). It has been shown in the literature that heat receptors as counter receptors can suppress itching sensations. The individual C-fibers perceive stimuli of a specific area of the skin, whereby a defined area of skin is innervated by a sensory cell. This region is called the receptive field. The receptive fields of C-fibers can partially overlap. Studies on humans through what is called micro-mapping discovered that the mechanically insensitive C-fibers have receptive fields of up to 5 cm² in size; the C-fibers that are mechanically sensitive are somewhat smaller and up to 2 cm² in size. Surprisingly, it was found that starting from a preferred treatment surface size of about 7 cm², it is possible to achieve particularly good response of the receptive fields of both types of C-fibers. Thus, with the preferred treatment size between 7 cm² and 18 cm² the receptive fields of the different types of C-fibers can be addressed in a particular efficient manner. In addition, the effect of the horizontally outflowing heat is compensated. Thereby itching, even of affected skin areas smaller than the treatment surface, can be treated surprisingly effectively. The embodiment constitutes a deviation from the prevailing opinion in the technical field, which considered the use of such large treatment surfaces as not feasible.

In a preferred embodiment, the size of the treatment surface is less than 1 cm². This has opened up a new field of application for devices used to treat itching.

In a preferred embodiment, the size of the treatment surface is less than 40 mm². Especially in the case of herpes diseases, especially in the mouth (so-called herpes labialis), the preferred maximum size of the treatment surface is ideal to cover all possible affected areas. In particular, a treatment surface of 20 mm² is suitable to cover all typical affected skin areas when the device is applied only once.

Furthermore, a herpes treatment device that exhibits such a treatment surface can be kept particularly compact. In this manner, device sizes corresponding to those of a lipstick can be achieved. Such a compact device is readily and willingly carried permanently on the body or in a carry-on bag, so that a treatment can be performed at any time. The permanent availability considerably increases the success of the treatment. The treatment surface is preferably round, a shape which is particularly suitable for the treatment of herpes, whose affected skin areas often have an organic, almost round shape.

In a preferred embodiment, the heating element heats the treatment surface to a treatment temperature of 50 to 65° C., preferably 55 to 60° C., wherein the treatment temperature is maintained for a treatment duration of 2 to 12 sec, preferably 3 to 6 sec, and wherein the size of the treatment surface is between 1 cm² to 18 cm². Here, several elements known per se have been combined into a novel combination that exhibits the surprising effect that even high treatment temperatures and large treatment surfaces cause surprisingly little pain or unpleasant sensations.

In a preferred embodiment, the heating element heats the treatment surface to a treatment temperature of 50 to 65° C., preferably 55 to 60° C., wherein the treatment temperature is maintained for a treatment duration of 2 to 12 sec, preferably 3 to 6 sec, and wherein the size of the treatment surface is at least 6 cm², preferably at least 7 cm². Statements in the technical literature regarding a combination of treatment temperatures, treatment duration and treatment surface have so far pointed in a different direction.

In a preferred embodiment, the heating element heats the treatment surface to a treatment temperature of 50 to 65° C., preferably 55 to 60° C., wherein the treatment temperature is maintained for a treatment time of 2 to 12 sec, preferably 3 to 6 sec, and wherein the size of the treatment surface is less than 1 cm².

The combination of treatment sizes increases the effectiveness of the device for the treatment of itching.

In a preferred embodiment, the heating element heats the treatment surface to a treatment temperature of 50 to 65° C., preferably 55 to 60° C., wherein the treatment temperature is maintained for a treatment duration of 2 to 12 sec, preferably 3 to 6 sec, and wherein the size of the treatment surface is less than 40 mm². Thereby a compact and at the same time effective device for the treatment of itching can be provided.

In a preferred embodiment, the heating element heats the treatment surface to a treatment temperature between 42° C. and 56° C., while maintaining the treatment temperature for a treatment duration of 2 sec to 20 sec and with a treatment surface size between 1 cm² and 18 cm². Such a device for the treatment of itching is particularly reliable in its use.

In a preferred embodiment, the heating element heats the treatment surface to a treatment temperature between 42° C. and 56° C., wherein the treatment temperature is maintained for a treatment duration of 2 sec to 20 sec and wherein the size of the treatment surface is at least 6 cm², preferably at least 7 cm². The development of skin treatment devices to date has taken a completely different direction and does not suggest this combination. However, the combination of treatment temperature, treatment duration and treatment surface can synergistically increase the effectiveness of the treatment in a manner that is greater than the known treatment efficiencies of the respective individual parameters would suggest.

In a preferred embodiment, the heating element heats the treatment surface to a treatment temperature between 42° C. and 56° C., while maintaining the treatment temperature for a treatment duration of 2 sec to 20 sec, and wherein the size of the treatment surface is less than 1 cm². This combination was a lucky choice, since from a large number of possible combinations of the sizes, the one that produced an unexpected treatment success was selected.

In a preferred embodiment, the heating element heats the treatment surface to a treatment temperature between 42° C. and 56° C., while maintaining the treatment temperature for a treatment duration of 2 sec to 20 sec, and wherein the size of the treatment surface is less than 40 mm². The combination represents a departure from the usual technically view.

In a preferred embodiment, the heating element heats the treatment surface to a treatment temperature of 49° C. to 53° C., while maintaining the treatment temperature for a treatment period of 3 to 20 seconds, and wherein the size of the treatment surface is between 1 cm² to 18 cm².

The combination of the described sizes in this embodiment surprisingly leads to the fact that several, even widely separated skin conditions, such as insect bites, can be treated at the same time. Even skin conditions that cover a larger area than the treatment surface can be effectively alleviated during a single treatment with these parameters.

In a preferred embodiment, the heating element heats the treatment surface to a treatment temperature of 49° C. to 53° C., whereby the treatment temperature is maintained for a treatment duration of 3 to 20 sec and whereby the size of the treatment surface is at least 6 cm², preferably at least 7 cm².

In the literature it was assumed that such a large treatment surface would not be effective for the treatment durations and temperatures disclosed.

In a preferred embodiment, the heating element heats the treatment surface to a treatment temperature of 49° C. to 53° C., while maintaining the treatment temperature for a treatment duration of 3 to 20 seconds and with a treatment surface size of less than 1 cm². The combination of the described sizes allows for a miniaturization of the device.

In a preferred embodiment, the heating element heats the treatment surface to a treatment temperature of 49° C. to 53° C., while maintaining the treatment temperature for a treatment period of 3 to 20 seconds, and the size of the treatment surface is less than 40 mm². Thus, further means for the treatment of itching and especially herpes can be provided.

In a preferred embodiment, the heating element heats the treatment surface to a treatment temperature between 40° C. and 65° C., wherein the treatment temperature is maintained for a treatment time of 4 sec to 6 sec and wherein the size of the treatment surface is between 1 cm² to 18 cm². By combining relevant sizes, a particularly high yield of the amount of heat introduced into the skin can be achieved.

In a preferred embodiment, the heating element heats the treatment surface to a treatment temperature between 40° C. and 65° C., wherein the treatment temperature is maintained for a treatment time of 4 sec to 6 sec and wherein the size of the treatment surface is at least 6 cm², preferably at least 7 cm². The combination of parameters allows for a particularly reliable treatment to be achieved.

In a preferred embodiment, the heating element heats the treatment surface to a treatment temperature between 40° C. and 65° C., while maintaining the treatment temperature for a treatment duration of 4 sec to 6 sec, and wherein the size of the treatment surface is less than 1 cm².

The combination of relevant sizes was a lucky choice by the inventors. Many possible combinations were available, while the improvement in the treatment of itching was not to be expected from this particular combination.

In a preferred embodiment, the heating element heats the treatment surface to a treatment temperature between 40° C. and 65° C., while maintaining the treatment temperature for a treatment time of 4 sec to 6 sec, and the size of the treatment surface is less than 40 mm². Due to the large temperature range disclosed herein and the small treatment surface in combination with a treatment duration of 4 to 6 seconds, a wide variety of skin conditions causing itching can be treated very flexibly.

In a preferred embodiment, the heating element heats the treatment surface to a treatment temperature between 42° C. and 53° C., particularly preferably between 50° C. and 53° C., wherein the treatment temperature is maintained for a treatment duration of 4 sec to 6 sec and wherein the size of the treatment surface is between 1 cm² to 18 cm². A device featuring this combination has proven to be surprisingly low-maintenance and robust. Even under suboptimal conditions, such as high humidity and heat, the device works surprisingly well.

In a preferred embodiment, the heating element heats the treatment surface to a treatment temperature between 42° C. and 53° C., particularly preferably between 50° C. and 53° C., wherein the treatment temperature is maintained for a treatment duration of 4 sec to 6 sec and wherein the size of the treatment surface is at least 6 cm², preferably at least 7 cm². In the embodiment the effectiveness of the treatment is particularly high.

In a preferred embodiment, the heating element heats the treatment surface to a treatment temperature between 42° C. and 53° C., particularly preferably between 50° C. and 53° C., wherein the treatment temperature is maintained for a treatment time of 4 sec to 6 sec and wherein the size of the treatment surface is less than 1 cm².

Such a device is governed by a high energy yield. Even low-power energy sources are sufficient to perform effective treatment.

In a preferred embodiment, the heating element heats the treatment surface to a treatment temperature between 42° C. and 53° C., particularly preferably between 50° C. and 53° C., wherein the treatment temperature is maintained for a treatment duration of 4 sec to 6 sec and wherein the size of the treatment surface is less than 40 mm². Thereby another alternative for the treatment of itching can be provided.

In a preferred embodiment, a hardware-implemented temperature monitor reversibly limits the maximum temperature of the treatment surface and a fuse switches off the device in case of a short circuit or unregulated continues heating. The maximum temperature relates preferably to the temperature that the treatment surface reaches at most during the treatment phase. The hardware-implemented temperature monitor is advantageous in ensuring that the maximum temperature is not exceeded. For the purpose of the invention, terms such as about, approximate, near or synonymous terms preferably denote a tolerance range of less than ±10%, preferably less than ±5%, particularly preferably less than ±1%. In the sense of the invention, a “hardware-implemented temperature monitor” preferably designates a temperature control system for the treatment surface, which is hardware-based and can switch off the power supply of the heating elements for the treatment surface. In particular, the “hardware-implemented temperature monitor” preferably allows to cut the power supply to the heating elements, when the maximum temperature is exceeded, independently of the regulation of the heating elements by the control device, e.g., the microprocessor. If, for example, a firmware is installed on the control device for the regulation of the heating elements, it is preferred that the hardware-implemented temperature monitor reliably limits the maximum temperature of the treatment surface even in case of a failure or a faulty execution of the firmware. In this manner it can be ensured particularly effectively by simple design means that the treatment surface of the device does not exceed a maximum temperature. Even in the event of malfunctions in the control device, for example after the ingress of liquids, the hardware-based temperature monitor can be used to ensure that the treatment surface does not exceed a maximum temperature at any time. This additional technical element for a temperature monitoring makes it possible to maintain an excellent safety standard without interfering with the operation of the hyperthermia treatment device.

As a further safety element, the device according to the invention comprises a safety fuse, which interrupts the power supply to the device in case of a short circuit of the device or uncontrolled heating of the device. In the sense of the invention, a safety fuse is preferably understood to be an overcurrent protection device, in which, for example, a circuit can be interrupted by the melting of a fusible conductor as soon as the current intensity exceeds a limit value over a time to be determined. It is preferred that the safety fuse is present in the device between the supply voltage being fed into the device and the device itself. In case of a malfunction, which is characterized by an uncontrolled high current flowing from the power supply into the device, the safety fuse will advantageously shut down the power supply to the device completely. A safety fuse offers a sufficiently fast and extremely reliable protection.

It has been shown that even with a faultless design of the device and the provision of a hardware-implemented temperature monitor, it cannot be excluded that, in extremely rare cases, an unregulated continuous heating of the heating elements occurs due to incorrect operation or damage to the device.

In the sense of the invention, continuous heating of the heating elements is preferably understood to mean that the temperature of the heating elements rises uncontrolled, i.e. not by a temperature-based control with the control device. If the hardware-implemented temperature monitor fails in these cases, the treatment surface can rise uncontrolled to temperatures far above the desired treatment temperature, for example to temperatures well above 65° C.

Although such faulty heating occurs extremely rarely, it can cause serious damage to the user. This is especially due to the fact that the skin areas to be treated hyperthermally are usually particularly sensitive and are characterized by redness, swelling or even wound formation. A significantly increased temperature above 65° can locally cause severe pain and skin burns at these sites.

In view of the special circumstances of the use of the device and the associated safety requirements, the safety fuse mentioned above is particularly advantageous in order to guarantee that the heating of the treatment surface is shut down even in the most unlikely instance of a malfunction. The fuse prevents excessive heating of the treatment surface, regardless of any temperature measurement, sensitive to defective temperature sensors. It was recognized that the power supply of the device represents a central regulatory interface that meets the highest safety requirements. By integrating the safety fuse into the current feed for supplying the device, it can be ensured that a maximum supply current is not exceeded during a certain time. Since continuous and uncontrolled heating of the heating elements above the desired temperature are related to an increased current flow for supplying the device, overheating of the treatment surface can be prevented in a particularly reliable manner. The current control allows for a particular fast reaction before the current is effective long enough to produce an elevated temperature corresponding to its strength. A safety mechanism as a final means based purely on temperature may not be fast enough as a result of the thermal inertia of the components involved.

The combined use of a hardware-implemented temperature monitor and a safety fuse is particularly advantageous for the device according to the invention.

A drawback of the safety fuse is that following the single triggering it permanently disconnects the supply voltage from the device Resumption of the use of the device following triggering of the safety fuse requires repair by a technician, for example replacement of the safety fuse. In terms of cost, the device has generally become unusable when the fuse has been triggered.

Advantageously, however, the hardware-implemented temperature monitor is set such that it does not need to cause permanent shutoff of the power supply to the device. Instead, the hardware-implemented temperature monitor is designed in such a way that if the temperature of the treatment surface exceeds a maximum temperature, the power supply to the heating elements is interrupted during the time period exceedance. Thus, the current interruption by the hardware-implemented temperature monitor is advantageously reversible, i.e., as soon as the temperature of the treatment surface again drops below the maximum temperature, the heating elements can heat again.

Thus, even after a one-time occurrence of a malfunction, the normal use of the device can be continued. The user might not notice the malfunction, because as a result of the selected maximum temperature, the effectiveness and the independence of the hardware-implemented temperature monitor no temperatures perceived by the user as unpleasant will develop.

The combination of the safety features of a hardware-implemented temperature monitor with a safety fuse allows a surprisingly reliable temperature control with the most economical means possible because of the hierarchy of safety barriers. Such a combination of additional safety barriers is particularly useful for a device that is connected to a mobile device. Mobile devices can be exposed to technical problems which comprise a safe control. For example, mobile devices can be affected by computer viruses. In such an instance it is a particular advantage when the device comprises inherent safety mechanism, preferably as doubling of safety barriers.

A further synergistic effect of combining the safety features of a hardware-implemented temperature monitor with a safety fuse can be seen in the fact that an unlikely but possible one-time failure of the control device is reversibly intercepted by the hardware-implemented temperature monitor. If, however, an extremely unlikely major problem should occur, which extends to the hardware-implemented temperature monitor, the safety fuse will take action as a last resort for a protection. Since the action of the fuse is irreversible, no potentially endangering further use by the user may take place under these circumstances. Instead a visit to a technician or to a specialized store is to be arranged.

Preferably, the temperature of the treatment surface is already controlled by means of the control device. If the control device fails due to e.g. an error occurring in the electronics, the hardware-implemented temperature monitor allows the heating elements to be shut down independently of the control device. Even if the control device were to fail in such a way, a safety fuse would not be triggered. Only in the extremely rare case that both the control device and the hardware-implemented temperature monitor fail, for example if the corresponding components are damaged, the safety fuse guarantee a final safety control element. If an increased current demand of the heating elements occurs in the process of a strong heating up, the safety fuse cuts off the entire power supply to the device. The hierarchy of safety barriers makes it possible to intercept a one-time malfunction of the control device extremely safely. The hardware-implemented temperature monitor intervenes unnoticed and rapidly without affecting the usability of the device. An even higher safety level can be achieved by the downstream safety fuse, so that the user can be provided with an immensely effective and safe treatment device.

By connecting the safety barriers in series, it can be surprisingly ensured that the treatment surface does not reach a temperature range that could endanger the patient.

In a preferred embodiment, the maximum temperature is between 54° C. and 58° C., preferably about 56° C. Surprisingly, it has been shown that a maximum temperature between 54° C. and 58° C., preferably about 56° C., does not jeopardize the success of the hyperthermic treatment, nor does it create an unpleasant sensation on the skin, so that this maximum temperature is an ideal first safety level to protect against overheating.

In a preferred embodiment, the hardware-implemented temperature monitor comprises at least a second temperature sensor for measuring the temperature of the treatment surface and a comparator, wherein the comparator compares the temperature of the treatment surface with the maximum temperature and if the maximum temperature is exceeded, stops the current feed to the at least one heating element.

In the sense of the invention, a comparator preferably denotes an electronic circuit for comparing two voltages, whereby the output indicates in binary form which of the two voltages is higher. In the prior art, various comparators are sufficiently well known, which are suitable for using two analog voltages to output one binary output signal and indicating which of the input voltages is higher. The Schmitt trigger may be mentioned as an example of a comparator circuit. It is preferred for a reference value for a voltage be applied to one input of the comparator using a voltage splitter. This reference value preferably corresponds to the voltage value that the second temperature sensor would show if the temperature of the treatment surface is equal to the maximum temperature. At the second input of the comparator, the output voltage of the temperature sensor, which depends on the temperature of the treatment surface, is preferably present. A particularly preferred temperature sensor has an NTC thermistor, i.e., a thermal resistor. This has a negative temperature coefficient, so that when the temperature increases, the resistance decreases and a higher current flows. PTC thermistors can also be used, which have a positive temperature coefficient, so that when the temperature increases, the resistance increases and a lower current flows.

If the temperature of the treatment surface rises, the voltage value at the comparator, regulated by the second temperature sensor, moves toward the voltage reference value that corresponds to the maximum temperature. As soon as the temperature exceeds the maximum temperature, the output signal on the comparator changes in a binary manner. The comparator is preferably integrated in the power supply of the heating elements. In other words, before the temperature of the treatment surface reaches the maximum temperature, the comparator preferably unblocks the supply voltage of the heating elements. However, as soon as the temperature is higher than the maximum temperature, the outlet of the comparator shuts off and interrupts the power supply to the heating elements. When the temperature of the treatment surface drops again, supply voltage is advantageously unblocked again by the comparator. As a result, reversible on and off switching of the heating elements can only take place for a time period during which the temperature of the treatment surface exceeds the maximum temperature. In addition, it may be preferred for the comparator to be unlocked by the control device when the device is turned on. Thus, if correct a start-up of the device does not take place, the comparator is configured in the setup phase to interrupt the current feed of the heating elements.

The preferred embodiment of the hardware-implemented temperature monitor described above has proven in tests to be particularly robust and reliable. Due to the reversibility of the safety switch and the simple design, the preferred embodiment is also characterized by low manufacturing and maintenance costs.

Due to the design independent of the control device and to the dedicated temperature sensor, reliable operation can be guaranteed even in the case of failure of a component of the control device. Similarly, a hardware-implemented temperature monitor in the described form using a comparator is particularly fast, since comparators are widely used electronic components that are characterized by their reliability and fast switching capability. For example, comparators with switching times of nanoseconds or less are available. Surprisingly, it was found that by using comparators in the circuit, due to their speed a particularly effective protective mechanism against overheating of the treatment surface was established.

In a preferred embodiment, the safety fuse has a threshold value for a maximum current corresponding to the heating of the treatment surface to 65° C. for 1 second. Tests have shown that only a temperature increase of more than 65° C. for more than 1 second is to be considered very critical for the sensation of pain and may lead to damage to the skin areas. It is advantageous that by adjusting the safety fuse to these parameter values, the safety fuse is not triggered prematurely in the case of a subcritical temperature increase of the treatment surface. In this manner, economic efficiency can be increased without having to make a compromise in terms of safety. Based on the electrical parameters of the heating elements, the person skilled in the art knows which safety fuse should be selected to guarantee the specified values. For this purpose, the current flow may be measured while simultaneously measuring the temperature of the treatment surface. In addition, it is particularly preferred to use a fast-acting safety fuse, which preferably reacts to a current increase within less than 20 ms. Thus, it was recognized that even a short-term increase in the current for less than 20 ms can lead to a temperature elevation for more than 1 second because of the thermal inertia of the treatment surface.

Compared with non-resettable, purely temperature-dependent thermal fuses, which likewise function by melting, the current-dependent safety fuse used here has several advantages. In the case of non-resettable, purely temperature-dependent thermal fuses, the melting does not take place upon application of a current above a threshold value, but only upon application of an external temperature that is higher than a defined maximum temperature. Thus in contrast to non-resettable, purely temperature-dependent thermal fuses, current-dependent safety fuses can react even before a certain undesirable temperature is reached as a result of an elevated current acting for a relatively long period. Likewise, non-resettable, purely temperature-dependent thermal fuses always require a certain reaction time in the presence of an external temperature above a defined maximum temperature. In this way, dangerous further temperature elevations can occur. In contrast to this, current-dependent safety fuses react more quickly and with minimal system-related latency times.

In a preferred embodiment, the threshold value of the fuse is preferably between 1 A and 2.5 A, particularly preferably about 2 A. Tests have shown that with regard to the preferred heating elements, the above-mentioned threshold values guarantee with especially good reliability that the temperature of the treatment surface does not exceed a temperature of 65° C. to 70° C. for more than 1 second. By melting of the safety fuse above current values of 1 A to 2.5 A, it can thus be ensured that the temperature of the treatment surface cannot enter a range that would be hazardous to health. Thus, in the case of a normal treatment, a normal treatment current that is less than 2.5 A, preferably 1 A occurs. If a malfunction occurs, e.g., in case of continuous heating, an increased current will flow. In this case, the fuse intervenes and effectively prevents uncontrolled heating.

The advantageous selection of the maximum temperature of the hardware-implemented temperature monitor to a value between 54° C. and 58° C., preferably of about 56° C., also ensures that a large enough distance is kept to a temperature at which the fuse trips as a result of a current value above the threshold value. In this manner, accidental triggering of the safety fuse, which would result in at least a replacement of the fuse, can be avoided as long as no major malfunctioning occurs, which includes the hardware-implemented temperature monitor.

In a further aspect, the invention relates to a system of device and mobile device, wherein the device is configured for a power supply by the mobile device and/or for data exchange with the mobile device and/or for control by the mobile device via an interface.

It may be preferred that a control device is included in the device, but it may also be preferred that the mobile device includes the control device, especially when controlled by the mobile device via an interface.

The average person skilled in the art recognizes that technical features and advantages of preferred embodiments of the device according to the invention also apply to the system according to the invention.

1. Device for hyperthermal treatment of itching comprising at least one treatment surface which can be heated by heating at least one heating element to a treatment temperature of 40° C. to 65° C. and can be controlled by a control device,
characterized in that
the device exhibits at least one interface and is configured for a power supply to the device by a mobile device and/or for data exchange with a mobile device and/or for control by a mobile device via the interface.

2. Device according to claim 1
characterized in that
the device comprises the control device.

3. Device according to claim 2,
characterized in that
the device is configured for parameterization of the control device by the mobile device.

4. Device according to claim 2 or 3,
characterized in that
the device is configured for transmission of a control program of the control device by the mobile device.

5. Device according to claim 1,
characterized in that
the mobile device includes the control device.

6. Device according to one or more of the previous claims,
characterized in that
the power supply of the device by the mobile device and/or the data exchange with the mobile device and/or the control by the mobile device via the interface is wireless.

7. Device according to one or more of the previous claims,
characterized in that
the treatment temperature is 40° C. to 60° C. and the size of the treatment surface is between 1 cm² and 18 cm².

8. Device according to one or more of the previous claims,
characterized in that
the treatment temperature is 40° C. to 60° C. and the size of the treatment surface is less than 1 cm².

9. Device according to one or more of the previous claims,
characterized in that
the device comprises an energy storage unit, preferably an accumulator, particularly preferably a lithium polymer accumulator and/or a metal hydride accumulator.

10. Device according to claim 9, wherein the accumulator is a solid-state accumulator, preferably a lithium ceramic accumulator.

11. Device according to one or more of the previous claims
characterized in that
the device comprises at least one first temperature sensor for measuring the temperature of the treatment surface, and the regulating device and/or control device adjusts the temperature of the at least one heating element based on the measurement data of the temperature sensor.

12. Device according to one or more of the previous claims, wherein a treatment temperature is maintained for a treatment duration of 1-10 sec.

13. Device according to one or more of the previous claims, wherein a hardware-implemented temperature monitor reversibly limits the maximum temperature of the treatment surface and a fuse switches off the device in case of a short circuit or uncontrolled continues heating.

14. Device according to claim 13, wherein the hardware-implemented temperature monitor comprises at least a second temperature sensor for measuring the temperature of the treatment surface and a comparator, wherein the comparator compares the temperature of the treatment surface with the maximum temperature and, if the maximum temperature is exceeded, cuts off the power supply to the heating element.

15. System comprising device and mobile device, wherein the device is configured for a power supply by the mobile device and/or for a data exchange with the mobile device and/or for a control by the mobile device via an interface.
